(19) United States
(12) Patent Application Publication
(10) Pub. No.: US 2019/0015132 A1
(43) Pub. Date: Jan. 17, 2019

(54) DEVICE AND METHOD OF USE FOR A VACUUM ASSISTED SPECIMEN RETRIEVAL BAG WITH A SEALED LAPAROSCOPIC ACCESS PORT

(71) Applicant: Matthew Roznovsky, New Braunfels, TX (US)

(72) Inventor: Matthew Roznovsky, New Braunfels, TX (US)

(21) Appl. No.: 16/036,835

(22) Filed: Jul. 16, 2018

Related U.S. Application Data
(60) Provisional application No. 62/532,453, filed on Jul. 14, 2017.

(51) Int. Cl. A61B 17/34; A61B 1/313; A61B 17/02

(52) U.S. Cl. CPC A61B 17/3423; A61B 1/3132; A61B 2017/00561; A61B 17/0218; A61B 2017/0225; A61B 17/0293

(57) ABSTRACT

Disclosed herein is a device and method of use for specimen retrieval bags and wound protectors. More specifically, the present invention generally relates to a device and method of use for a specimen retrieval bag that utilizes vacuum assistance in conjunction with a sealed laparoscopic access port wound protector. In one embodiment the wound protector device is comprised of ring portions and a bladder portion. In other embodiments, the device is comprised of a bladder opening tube portion. In yet other embodiments, the bladder portion is further comprised of horizontal ridges and vertical ridges. In an embodiment, the specimen retrieval bag device is comprised of a handle portion and a retrieval bag portion. In another embodiment, the specimen retrieval bags handle portion is further comprised of a lumen portion, a pump lever portion, rubber gasket portions, metal prong portions, and an extension/retraction lever.

### CROSS-REFERENCE TO RELATED APPLICATIONS

This application claims the benefit under Title 35 United States Code § 119(e) of U.S. Provisional Patent Application Ser. No. 62/532,453; Filed: Jul. 14, 2017, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention generally relates to a device and method of use for specimen retrieval bags and wound protectors. More specifically, the present invention generally relates to a device and method of use for a specimen retrieval bag that utilizes vacuum assistance in conjunction with a sealed laparoscopic access port wound protector.

### BACKGROUND OF THE INVENTION

Without limiting the scope of the disclosed device, the background is described in connection with a novel device and method of use for a specimen retrieval bag that utilizes vacuum assistance in conjunction with a sealed laparoscopic access port wound protector.

To help alleviate these issues, several approaches in the prior art are utilized. The following examples are some of the common ones utilized today.

As a first example, U.S. Pat. No. 9,138,137 issued to Vivek R. Deshmukh et al. on Sep. 22, 2015 is directed to an inflatable retractor. While the retractor allows the adjustment of the outer wall of the retractor to be expanded, it is rigid and the interior lumen or cavity portion expands along with the outer wall. That is the interior lumen or cavity is not sealed allowing the defect to be exposed.

As a second example, U.S. Pat. No. 9,101,354 issued to Jeremy J. Albrecht et al. on Aug. 11, 2015 is directed to a wound retractor with gel cap. While this is a base wound protector utilized and is common in the art, the interior lumen or cavity portion remains open and does not seal, also allowing the defect to be exposed.

As a third example, U.S. Pat. No. 8,491,471 issued to Vivek R. Deshmukh et al. on Jul. 23, 2013 is directed to an inflatable surgical retractor. While this device allows the inflation of the surgical retractor, the interior lumen or cavity is also not sealed, still allowing the defect to be exposed.

While all of the aforementioned devices may fulfill their unique purposes, none of them fulfill the need for a practical, effective, and efficient means for specimen retrieval.

Therefore, the present invention proposes a novel device and method of use for a specimen retrieval bag that utilizes vacuum assistance in conjunction with a sealed laparoscopic access port wound protector to address the shortcomings in the prior art.

### BRIEF SUMMARY OF THE INVENTION

The present invention, therefore, provides for a specimen retrieval bag and wound protector. More specifically, the present invention generally relates to a device and method of use for a specimen retrieval bag that utilizes vacuum assistance in conjunction with a sealed laparoscopic access port wound protector.

The wound protector device or sealed laparoscopic access port device provides access to the abdominal cavity while providing a circumferential wound protector on the outside. The device assists in maximizing a minimal opening/bodily defect during surgical procedures. The device is see as a safe and efficient reliable alternative to morcellation. In one embodiment the device is comprised of ring portions and a bladder portion. In other embodiments, the device is comprised of a bladder opening tube portion. In yet other embodiments, the bladder portion is further comprised of horizontal ridges and vertical ridges. The combination of internal CO₂ (pneumo) pushing against the pull of displaced fluid in the bladder portion allows the surgeon to retrieve larger objects/products than previously through the same size defect. The ring portions anchor the device inside and outside the body. The bladder portion when filled occludes air and fluid from travelling through the device. The bladder portion allows the user of the device to manipulate filtration of products going in and out (through) the device while still conserving pneumo within the patient through the procedure.

In an embodiment, the specimen retrieval bag device is comprised of a handle portion and a retrieval bag portion. In another embodiment, the specimen retrieval bags handle portion is further comprised of a lumen portion, a pump lever portion, rubber gasket portions, metal prong portions, and an extension/retraction lever. The specimen retrieval bag device may be used in conjunction with the sealed laparoscopic access port device to extract product or large specimens from the human body during laparoscopic surgical procedures as a safe and efficient alternative to morcellation.

In summary, the present invention discloses a novel device and method of use for a specimen retrieval bag that utilizes vacuum assistance in conjunction with a sealed laparoscopic access port wound protector.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a perspective view of the sealed laparoscopic access port device in accordance with embodiments of the disclosure;

FIG. 2A is a front view of the sealed laparoscopic access port device in an extended configuration in accordance with embodiments of the disclosure;

FIG. 2B is a front view of the sealed laparoscopic access port device in a collapsed position in accordance with embodiments of the disclosure;

FIG. 3 is a top view of the sealed laparoscopic access port in accordance with embodiments of the disclosure;

FIG. 4A is a sectional side view of the sealed laparoscopic access port device in accordance with embodiments of the disclosure;

FIG. 4B is a sectional side view of the sealed laparoscopic access port device illustrating the entry of the vacuum assisted specimen retrieval bag in accordance with embodiments of the disclosure;

FIG. 4C is a sectional side view of the sealed laparoscopic access port device illustrating the pass through of the vacuum assisted specimen retrieval bag in accordance with embodiments of the disclosure;

FIG. 5 is a perspective view of the sealed laparoscopic access port device illustrating the fluid control line in accordance with embodiments of the disclosure;

FIG. 6 is a side view of the vacuum assisted specimen retrieval bag device illustrated in a closed configuration in accordance with embodiments of the disclosure;

FIG. 7 is a side view of the vacuum assisted specimen retrieval bag device illustrated in an extended configuration in accordance with embodiments of the disclosure;

FIG. 8 is an environmental view of the vacuum assisted specimen retrieval bag with a sealed laparoscopic access port device in accordance with embodiments of the disclosure;

FIG. 9 is an environmental view of the vacuum assisted specimen retrieval bag shown in the expanded position in accordance with embodiments of the disclosure;

FIG. 10 is an environmental view of the vacuum assisted specimen retrieval bag shown in the non fully expanded position in accordance with embodiments of the disclosure.

### DETAILED DESCRIPTION OF THE INVENTION

Disclosed herein is an improved device and method of use directed to a vacuum assisted specimen retrieval bag with a sealed laparoscopic access port device. The numerous innovative teachings of the present invention will be described with particular reference to several embodiments (by way of example, and not of limitation).

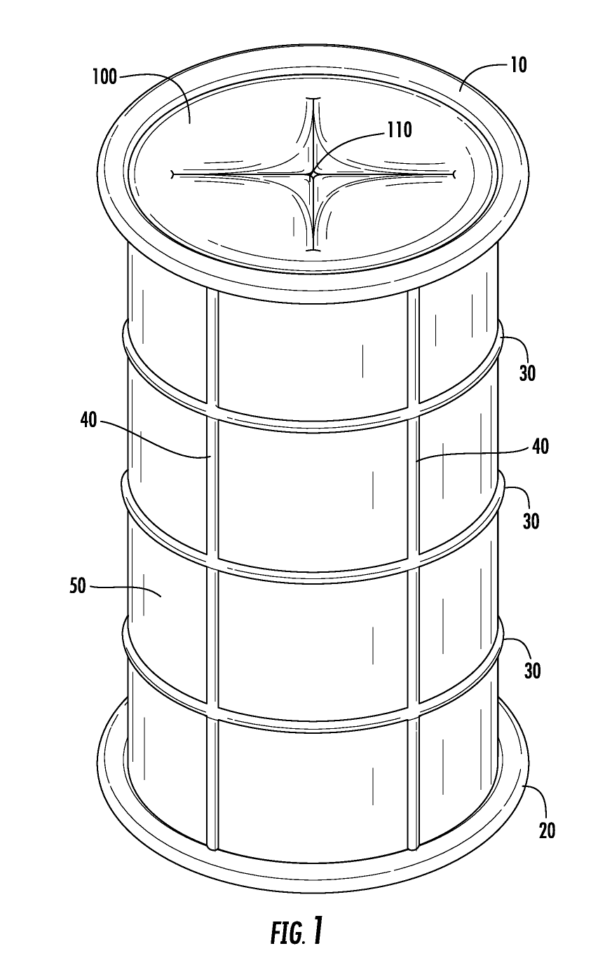

Reference is first made to FIG. 1, a perspective view of the sealed laparoscopic access port device or wound protector device in accordance with embodiments of the disclosure. In one embodiment the wound protector device is comprised of ring portions 10, 20 and a bladder portion 100. The ring portions 10, 20 are located at the top and bottom end portions of the device and are made of a rigid material such as but not limited to a polymer. In embodiments, the ring portions 10, 20 are circular. The bladder portion 100 extends from the top ring portion 10 to the bottom ring portion 20 and is made of a flexible material. The bladder portion may be filled and unfilled with a material such as but not limited to a liquid or gas. In embodiments the liquid is water. By filling and un-filling the device, the device center portion 110 is opened and closed. In embodiments, the bladder portion 100 is made of polyurethane. In other embodiments, the bladder portion 100 is latex free.

In yet other embodiments, the bladder portion is further comprised of horizontal ridges 30 and vertical ridges 40 which are made of rigid materials to provide further strength and support to the bladder portion 100 of the device. The horizontal ridges 30 provide traction and friction to help keep the device in place while inside the body.

Also illustrated in this figure is the bladder portion's 100 top opening 110.

The device functions as a wound protector/retractor that provides 360 degrees of radial retraction; while also providing an internal bladder portion 100 for full closure of device from all outside elements. The device also maintains moisture at the incision site and reduces surgical site infections. This is accomplished by preventing direct contact, airborne contact, and self-contamination to the surgical site incision. The impermeable bladder portion 100 creates a barrier of the wound margins and/or the environment.

The device assists in freeing up hands, eliminating point trauma, protects wound edges from contamination, has an internal bladder portion 100 that can inflate/deflate to close passageway to internal body, and also aids in retrieval of specimens through gravity and pressure working together with the fluid or air filled internal bladder portion 100.

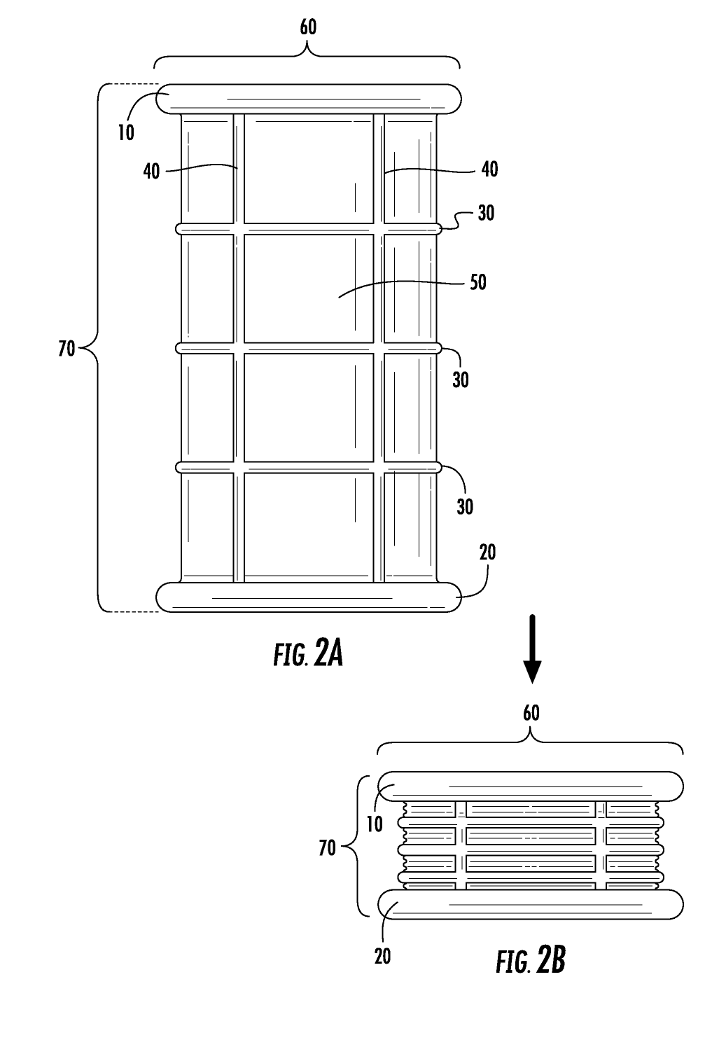

Reference is next made to FIG. 2A, a front view of the sealed laparoscopic access port device in an extended configuration in accordance with embodiments of the disclosure. In this illustration the device is shown in an extended or elongated configuration. The ring portions 10, 20 will have a diameter 60 and bladder portion length 70 (measured in its' elongated state). The device may be made in various sizes to accommodate various defect size applications. As an example and not a limitation, two sizes can be made for operating room applications. One size can be made to fit a 5-9 mm surgical incision. The ring portions 10, 20 will have a diameter 60 of 11 mm with the bladder portion 100 having a length 70 of 9.5 mm. Another size can be made to fit a 9-14 mm surgical incision. The ring portions 10, 20 will have a diameter 60 of 15 mm with the bladder portion 100 having a length 70 of 14.5 mm. In other embodiments, the upper ring portion 10 and the lower ring portion 20 may have different diameters 60. That is the upper ring portion 10 may have a diameter 60 that is different than the lower ring portion 20 diameter 60.

Reference is now made to FIG. 2B, a front view of the sealed laparoscopic access port device in a collapsed position in accordance with embodiments of the disclosure. Illustrated in this view is the device in a collapsed or compressed configuration.

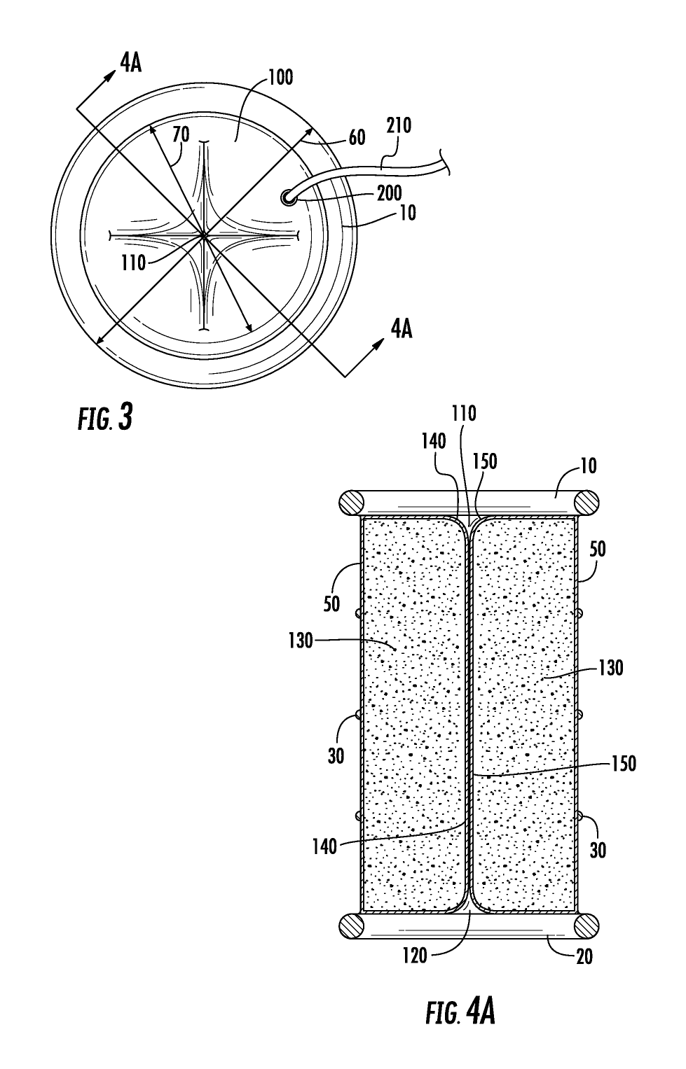

Reference is next made to FIG. 3, a top view of the sealed laparoscopic access port in accordance with embodiments of the disclosure. Illustrated in this figure is the top opening portion 110 of the bladder portion 100. Also shown is the upper rings' 10 inside diameter 70 and the outside diameter 60. Illustrated in this figure is the device with the bladder portion 100 filled, sealing or closing the top opening portion 110. The top opening portion 110 is an opening that extends the length 70 of the device or bladder portion 100 from the top to the bottom through the center 110 of the bladder portion 100.

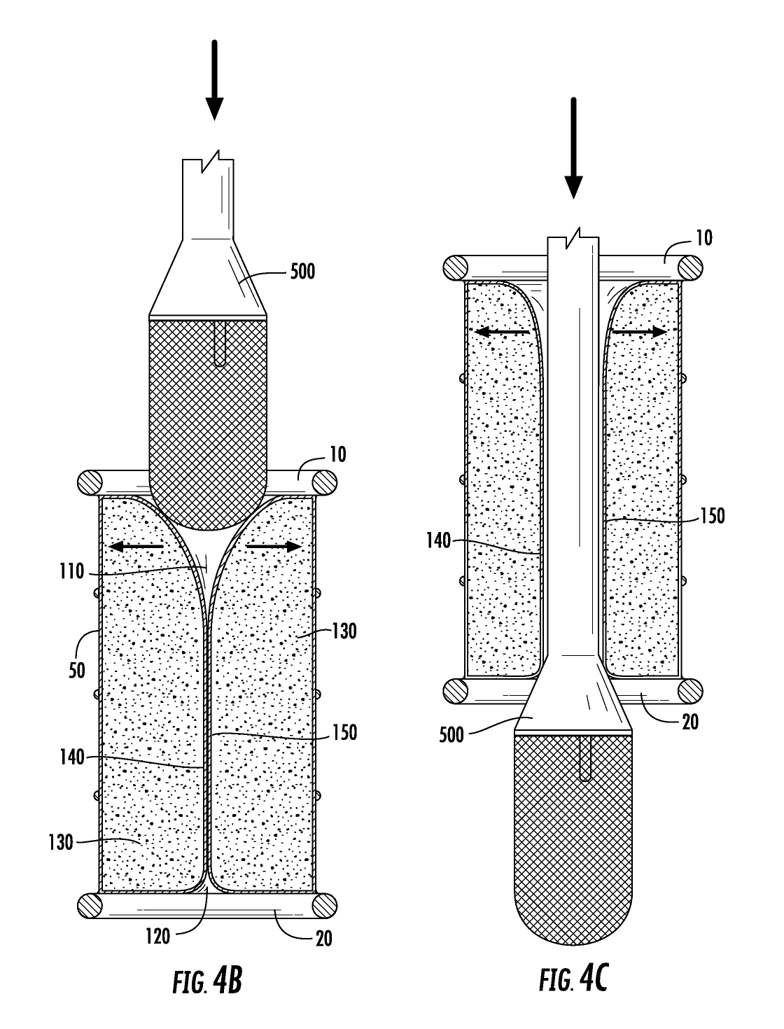

Reference is now made to FIG. 4A, a sectional side view of the sealed laparoscopic access port device in accordance with embodiments of the disclosure. Illustrated in this figure is the bladder portion 100 filled with a material 130 closing the center opening portion from the top opening portion 110 to the bottom opening portion 120. In this illustration identified is also a left side surface area 140 of the bladder portion 100 and a right side surface area 150 of the bladder portion 100.

Reference is next made to FIG. 4B, a sectional side view of the sealed laparoscopic access port device illustrating the entry of the vacuum assisted specimen retrieval bag in accordance with embodiments of the disclosure and FIG. 4C is a sectional side view of the sealed laparoscopic access port device illustrating the pass through of the vacuum assisted specimen retrieval bag in accordance with embodiments of the disclosure. Illustrated in this figure is how the second component/device, specifically, the vacuum assisted specimen retrieval bag, is inserted and passed through the sealed laparoscopic access port device. Since the bladder portion 100 is filled with a fluid material such as a liquid or a gas, the bladder portion 100 may be compressed or deformed in the horizontal direction to allow the vacuum assisted specimen retrieval bag to pass through the center opening portion of the bladder portion 100.

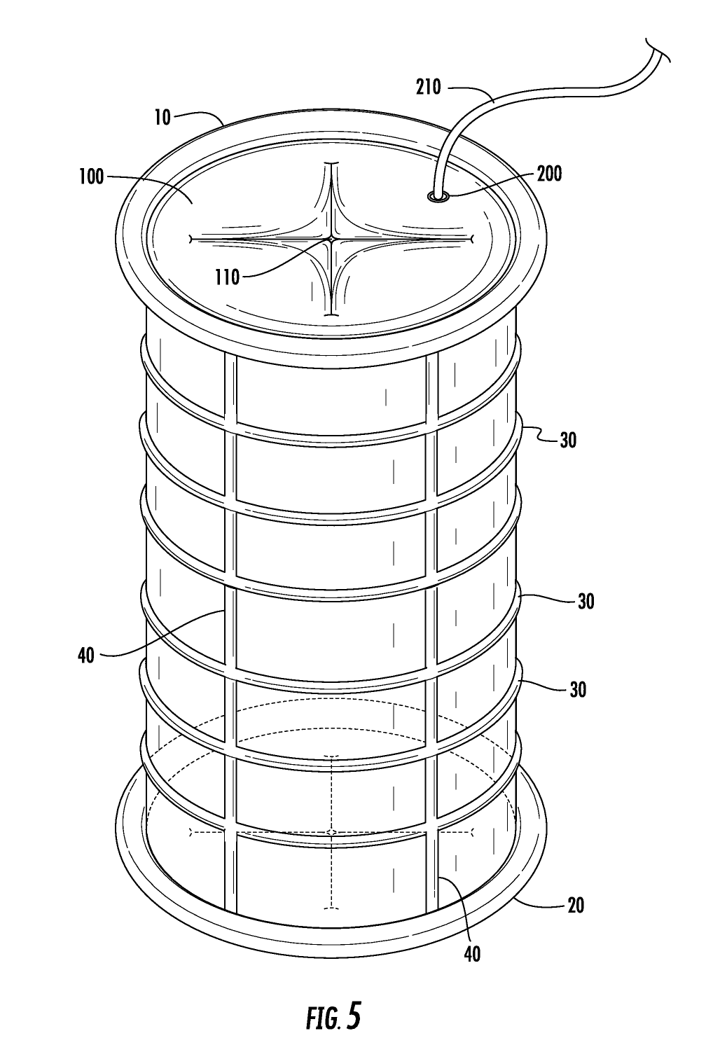

Reference is next made to FIG. 5, a perspective view of the sealed laparoscopic access port device illustrating the fluid control line in accordance with embodiments of the disclosure. In other embodiments, the device is further comprised of a bladder opening tube portion 200. The bladder opening tube portion 200 allows the bladder portion 100 to be filled and unfilled with a material such as but not limited to a liquid or gas. In embodiments the liquid is water. The bladder opening tube portion 200 becomes exposed once the ring portions 10, 20 are rolled down and activated. Fluid control of the bladder opening tube portion 200 is accomplished through a stop lock. The bladder opening tube portion 200 may have a tube 210 to help facilitate the filling of the bladder. In an embodiment the tube 210 may be connected to a mouth piece or syringe to help facilitate the filling of the bladder. In embodiments, the bladder may be filled by mouth or syringe.

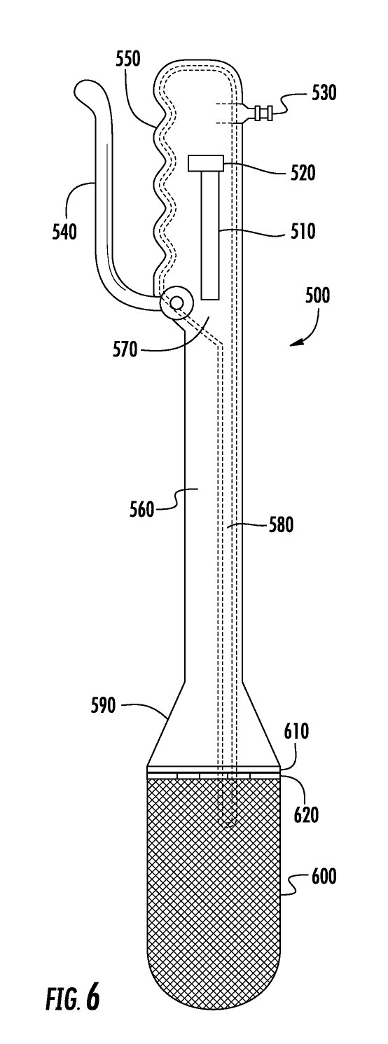

Reference is now made to FIG. 6, a side view of the vacuum assisted specimen retrieval bag device illustrated in a closed configuration in accordance with embodiments of the disclosure. In an embodiment, the specimen retrieval bag device is comprised of a handle portion 500 and a retrieval bag portion 600. In another embodiment, the specimen retrieval bags handle portion 500 is further comprised of a lumen portion 570, 580, a pump lever portion 540, rubber gasket portions 610, 620, metal prong portions, and an extension/retraction lever 520. In additional embodiments, the specimen retrieval bag handle portion 500 is further comprised of a grip portion 550, an extended portion 560, and a flare portion 590. The retrieval bag portion 600 in embodiments is made from a flexible material such as a polymer. In embodiments, the retrieval bag portion 600 is made of standard specimen bag material also comprised of threaded material such as rubber throughout the surface of the retrieval bag portion 600 for reinforcement. This configuration allows the retrieval bag portion 600 to stretch and contort to maximize a 360 degree python squeeze on the specimen or product being retrieved into a vertical cylinder shape for maximizing space during retrieval. The gasket portions 610, 620 allow an airtight seal between the specimen retrieval bag handle portion 500 and the retrieval bag portion 600. The lumen portions 570, 580 is a cavity that allows the specimen retrieval bag handle portion 500 to provide suction and hold material that has been suctioned, such as but not limited to bodily fluids. The pump lever portion 540 when squeezed, provides suction. The extension/retraction lever 520 slides back and forth along the extension/retraction lever track 510. Illustrated in this figure is the extension/retraction lever 520 in the closed position. When the extension/retraction lever 520 is slid down to the opposite end of the extension/retraction lever track 510, the device is in the extended or open configuration. That is the extension/retraction lever 520 controls the retraction and extension of the metal prong portions. Lastly, the specimen retrieval bags handle portion 500 may be further comprised of a release valve or port 530. This allows release of the air or material (such as excess fluid) from the lumen portions 570, 580.

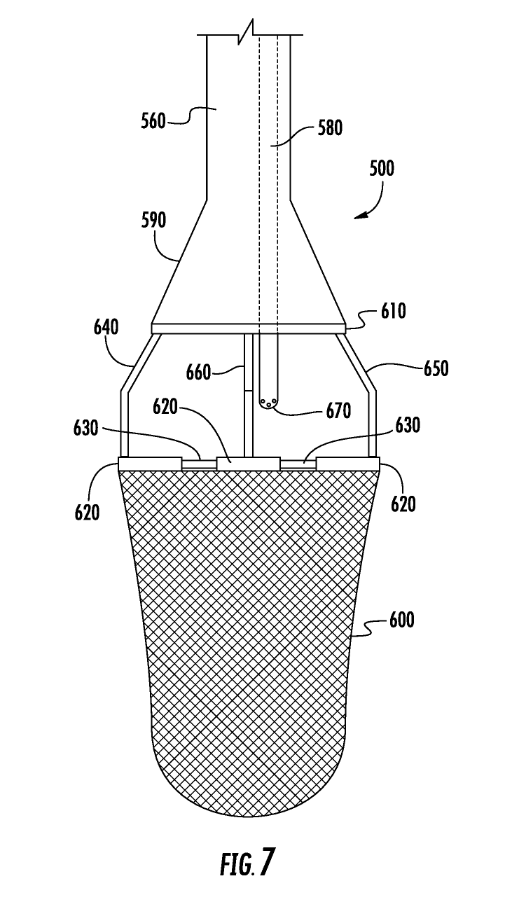

Reference is next made to FIG. 7, a side view of the vacuum assisted specimen retrieval bag device illustrated in an extended configuration in accordance with embodiments of the disclosure. Illustrated in this figure is the vacuum assisted specimen retrieval bag with the metal prong portions 640, 650, 660 extended allowing collection of a specimen in the retrieval bag portion 600. The vacuum assisted specimen retrieval bag device is extended once inside the body cavity. The metal prong portions 640, 650, 660 are extended at an angle to push the lower gasket portion 620 out laterally to provide more room for a large specimen and also to allow space between the upper gasket portion 610 and the lower gasket portion 620 to receive the product or specimen being collected into the retrieval bag portion 600. Also illustrated is the lower gasket portion 620 separating into segments to allow the specimen retrieval bag portion 600 to enlarge its' opening. Also shown is the lower gasket portion support ring 630. Lastly, also shown is the suction tip 670 of the handle portion 500. The suction tip 670 is comprised of small holes that provide vacuum and suction for the vacuum assisted specimen retrieval bag device. The suction tip 670 provides two functions: vacuum/suction to the retrieval bag and suction for excess fluids from the product or specimen being conformed to a vertical cylinder shape by the retrieval process. This excess fluid is stored in the in the lumen portions 570, 580. Once the specimen or product is retrieved while the vacuum assisted specimen retrieval bag device is in the extended position, the vacuum assisted specimen retrieval bag device may be closed or retracted. This creates an airtight seal by the gasket portions 610, 620 sealing with each other and allowing a vacuum suction assist of the retrieval bag by squeezing the pump lever portion 540.

The specimen retrieval bag 600 is configured in shape to a vertical cylinder to conform the specimen or product to be retrieved through a minimally non-invasive small bodily defect or cavity.

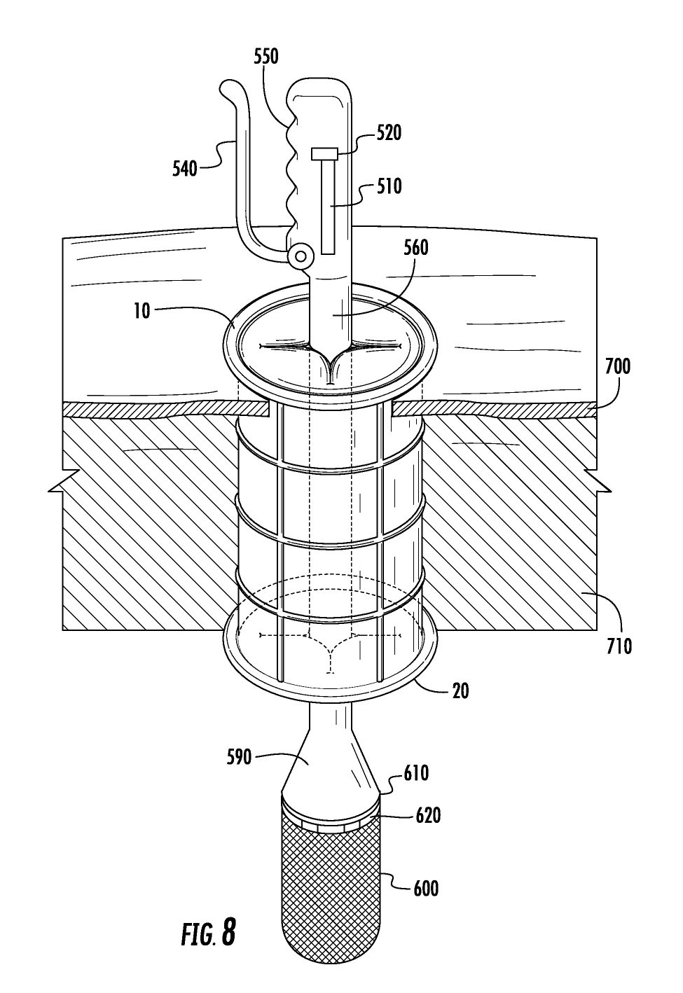

Reference is now made to FIG. 8, an environmental view of the vacuum assisted specimen retrieval bag with a sealed laparoscopic access port device in accordance with embodiments of the disclosure. Illustrated in this figure is the sealed laparoscopic access port device in conjunction with the vacuum assisted specimen retrieval bag device. The vacuum assisted specimen retrieval bag device is shown in the closed position passed through the sealed laparoscopic access port device.

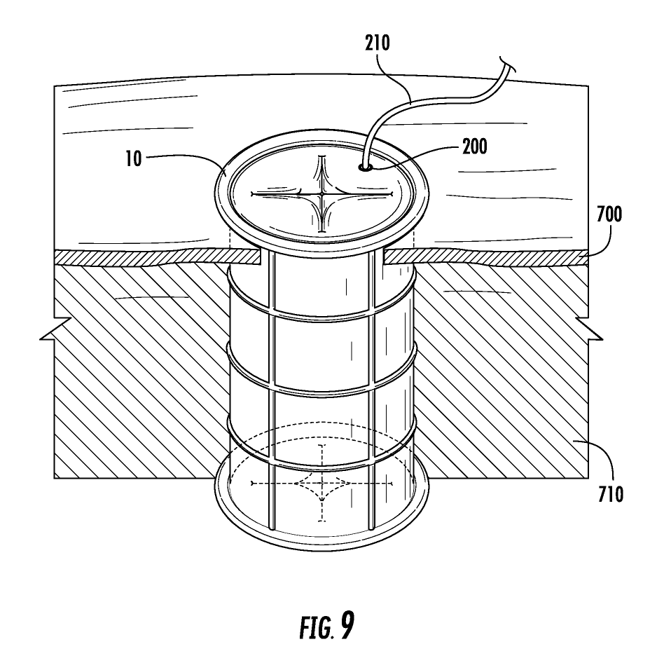

Reference is next made to FIG. 9 is an environmental view of the vacuum assisted specimen retrieval bag shown in the expanded position in accordance with embodiments of the disclosure. Illustrated here is the bladder portion in the expanded position sealing the access port.

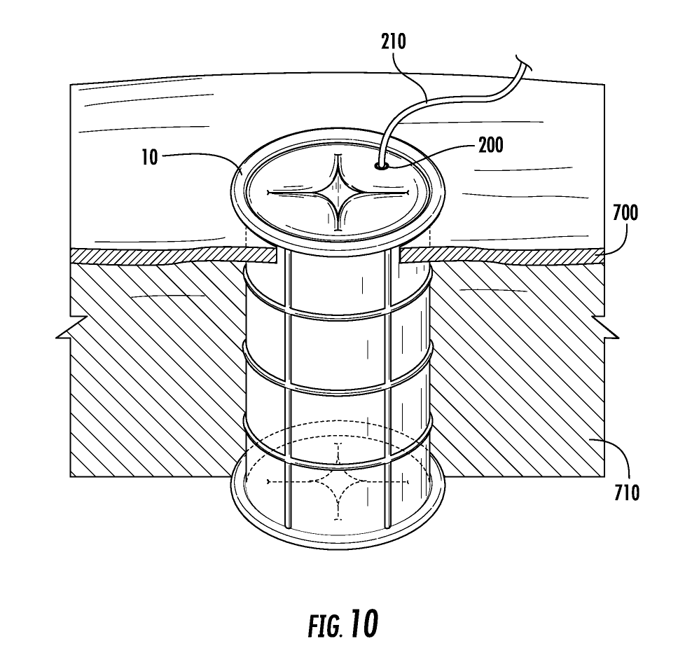

Reference is lastly made to FIG. 10 is an environmental view of the vacuum assisted specimen retrieval bag shown in the non fully expanded position in accordance with embodiments of the disclosure. Illustrated here is the bladder portion in the non fully expanded position allowing an opening in the center longitudinal axis of the bag.

In brief, the invention is directed to a device and method of use for a sealed laparoscopic access port device in accordance with embodiments of the disclosure.

The disclosed device and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method of use, except as may be outlined in the claims.

Alternative applications for this invention include using this device or method of use in any application where the collection of a specimen is needed. In addition, the use of the sealed laparoscopic access port may also be utilized as a wound filler or gunshot wound filler. The device could be used to fill and maintain bullet wounds. The device would plug the hole while still allowing internal access. The device completely seals and applies direct pressure to the wound. Different sizes could be manufactured for different wound sizes. That is, the sizing is appropriate for the defect. The device would be inserted into the defect and then filled. IN embodiments, to fill a 1-3 cm incision, opening, or bullet hole, the ring portions 10, 20 will have a diameter 60 of 6 cm with the bladder portion having a length 70 of 4 cm. Consequently, any embodiments comprising a one piece or multi piece device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications, references, patents, and patent applications mentioned in the specification are indicative of the level of those skilled in the art to which this invention pertains. All publications, references, patents, and patent applications are herein incorporated by reference to the same extent as if each individual publication, reference, patent, or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device and/or methods of use disclosed and claimed herein can be made and executed without undue experimentation in light of the present disclosure. While the device and methods of use of this invention have been described in terms of preferred embodiments, it will be apparent to those skilled in the art that variations may be applied to the device and/or methods of use and in the steps or in the sequence of steps of the method of use described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A device for a sealed laparoscopic access port as herein disclosed.

2. A device for a vacuum assisted specimen retrieval bag with a sealed laparoscopic access port as herein disclosed.
